(19) United States
(12) Patent Application Publication
(10) Pub. No.: US 2021/0177743 A1
(43) Pub. Date: Jun. 17, 2021

(54) Self-Assembling Multi-Domain Peptide Based Hydrogels

(71) Applicant: New Jersey Institute of Technology, Newark, NJ (US)

(72) Inventors: Vivek A. Kumar, Newark, NJ (US); Peter Nguyen, New Rochelle, NY (US); Patricia Iglesias-Montoro, Haledon, NJ (US); Biplab Sarkar, Newark, NJ (US); Victoria Harbour, East Brunswick, NJ (US); Zain Siddiqui, Paterson, NJ (US)

(73) Assignee: New Jersey Institute of Technology, Newark, NJ (US)

(21) Appl. No.: 17/267,876

(22) PCT Filed: Aug. 16, 2019
(86) PCT No.: PCT/US2019/046746

§ 371 Date: Feb. 11, 2021

Related U.S. Application Data
(60) Provisional application No. 62/765,157, filed on Aug. 17, 2018.

(51) Int. Cl. A61K 9/00; C07K 14/81; A61K 38/57; A61P 3/06; A61K 45/06

(52) U.S. Cl. CPC A61K 9/0024; C07K 14/811; C07K 2319/02; A61P 3/06; A61K 45/06; A61K 38/57

(57) ABSTRACT

An injectable peptide-based hydrogel is disclosed that incorporates a peptide inhibitor of proprotein convertase subtilisn/kexin type 9. The hydrogel is a polymer composed of the 13-amino-acid protein, Pep2-8, (TVFTSWEEYLDWV) attached to a self-assembling peptide of the ABA block structure (ESLSLSLSLSLSLEG) to generate the repeating multidomain peptide sequence (ESLSLSLSLSLSLEGTVFTSWEEYLDWV).

### CROSS-REFERENCE TO RELATED APPLICATION

The present application claims the benefit of the filing date of U.S. Provisional Patent Application No. 62/765,157, filed Aug. 17, 2018, the disclosure is hereby incorporated herein by reference.

### TECHNICAL FIELD

The present application discloses hydrogels that function as cholesterol lowering drugs. More specifically, there is disclosed a library of clinically relevant, injectable peptide based hydrogels that incorporate an inhibitor of the proprotein convertase subtilisin/kexin type 9.

### BACKGROUND

Cardiovascular disease (CVD), which includes disease of the heart, blood vessels and cerebral vasculature, accounts for more than one third of deaths worldwide. CVD has remained the primary cause of death for Americans since 1920. Over 40% of CVDs stem from failure to deliver enough blood to the body, called ischemic heart disease. Ischemia, constricted blood, is the result of atherosclerosis, a condition that occurs when a plaque containing LDL-c and other cellular debris clogs the arteries. Plaque formation begins when a blood vessel is damaged by risk factors such as smoking, genetics, or age that cause small lesions on the endothelial layer of the vessel. The lesions create an opening in the endothelium that allows LDL-c to enter. Over time, the trapped LDL-c aggregates, oxidizes and provokes an immune response. The attracted macrophages and T-cells release by-products that harden the plaque and contribute to its growth. The plaque restricts the area over which blood can flow, increasing shear force on the vessel walls. The force can rupture the plaque, causing small particles of broken clot to lodge in vessels of the brain and heart, which may result in stroke or heart attack.

Numerous studies have linked elevated levels of LDL-c, informally called ‘bad cholesterol’, in blood plasma to atherosclerosis (the buildup of fatty-plaque in arteries and veins) and ischemic cardiovascular disease (CVD). Proprotein convertase subtilisin/kexin type 9 (PCSK9) is a circulating serine protease, originating in the liver, that regulates the expression of low-density lipoprotein (LDL) receptors on liver cells. LDL receptors are critical to the digestion of LDL cholesterol (LDL-c) in blood plasma.

In cholesterol metabolism, the LDL receptor is expressed on the surface of hepatocytes (liver cells) where it binds to circulating LDL-c before the complex is drawn into the cell by endocytosis. Within the endosome, the LDL receptor releases LDL-c and adopts a closed position. Subsequently, lysosomes degrade LDL-c, effectively removing ‘bad cholesterol’ from circulation, and the LDL receptor is returned to the surface of the hepatocyte, where it opens, and resumes capturing extracellular LDL-c. In some cases, the LDL receptor binds to PCSK9 in addition to LDL-c at the cell surface. When the complex is drawn into the cell, PCSK9 binds tightly to the LDL receptor, locking the molecule in an open position. The receptor fails to close and the entire complex (PCSK9, LDL-c, and LDL receptor) proceeds to lysosomal degradation.

PCKS9, therefore, plays a key role in cholesterol metabolism. Lower concentrations of circulating PCSK9 increase the rate of LDL receptor recycling, in turn, greater receptor expression on hepatocytes decreases LDL-c in blood serum. Several studies have linked “loss of function” PCKS9 mutations in humans to significantly lower levels of circulating LDL-c. Individuals with genes encoding non-functional PCSK9 expressed average LDL-c levels of 100±45 mg/dl compared to normal LDL-c levels, 138±42 mg/dl. Additionally, no health abnormalities have been associated with non-functional PCSK9 mutations. The reduced cholesterol levels stemming from “loss of function” mutations may significantly impact CVD pathology and disease pathways. One study found individuals with non-functional PCSK9 were 88% less likely to develop cardiovascular disease than normal individuals. These findings have made PCSK9 inhibition an attractive biological target for treating high cholesterol associated with CVD.

The standard for affordable cholesterol drugs is currently statins, which block the enzyme responsible for producing cholesterol. Statins frequently illicit adverse effects (myopathy and increased risk of incident diabetes) or are completely ineffective. Concerns over adverse effects make doctors less likely to prescribe statins and patients more likely to discontinue their use.

A number of companies are known to be developing or have already released drugs designed to inhibit PCKS9. The majority, including Amgen, Sanofi/Regeneron, and Pfizer, developed humanized monoclonal antibodies (mAbs), injected as a serum, that directly inhibit PCKS9. Others, including Serometrix LLC and Shifa Biomedical Corp., are in preclinical development of small molecules and peptides, designed for oral delivery, that directly inhibit the protease. Small molecules and mimetic peptides are significantly less expensive to produce than mAb therapies.

Zhang et al. in the Journal of Biological Chemistry, 289(2), 942-955 (2014), found at http://doi.org/10.1074/jbc.M113.514067 identified a 13 amino-acid (TVFTSWEEYLDWV), linear peptide, Pep2-8, that is a positive regulator of the LDL receptor, thereby decreasing LDL concentration. Under normal conditions, PCKS9 initially binds to the LDL receptor by interacting with the receptor's epidermal-like growth factor domain (EGF-A). The bound PCKS9 holds the receptor ‘open’, flagging it for lysosomal degradation, instead of recycling the receptor back to the surface of the hepatocyte. Pep2-8 mimics the EGF-A domain of the LDL receptor, competitively binding to PCSK9, thereby preventing the convertase from binding to LDL receptors. Introducing Pep2-8 to hepatocytes with low LDL receptors due to PCSK9 has been shown to restore the receptors to the cell surface. However, the delivery of a small peptide is difficult without a proper vehicle, since small peptides are rapidly cleared in the body. Peptide-based and small molecule drugs that enter the body as pills or injected serums are often quickly degraded by proteases and enzymes. Degradation impacts the intended biological function of drug molecules, decreasing efficacy.

Administration of small molecule drugs is often a steep hurdle in drug development due to dissonance between pharmacokinetics (the body's effect on the drug) and pharmacodynamics (the drug's effect on the body). Penetration through biological barriers is critical for drugs targeting sites within the body (such as circulating PCSK9). Active and passive transporters on the epithelial cells, however, decrease penetration and the bioavailability of molecules, preventing otherwise effective drugs from reaching their target sites.

Accordingly, there is a need for non-allergenic cholesterol drugs for use in cardiovascular disease treatment. Drugs that reduce cholesterol can act both as a preventative measure for groups at risk for CVD as well as a treatment for individuals already suffering from CVD conditions. Furthermore there is a need for a delivery system to overcome the obstacles of administration of such small molecule drugs.

### SUMMARY OF THE INVENTION

The present invention solves the problems of current state of the art and provides many more benefits. The disclosure describes a small molecule, hydrogel therapy targeted to PSCSK9 inhibition. Specifically, it describes a self-assembling multidomain peptide with the sequence for Pep2-8 that crosslinks to form a hydrogel, which has better targetability, persistency, and can activate more receptors. The resulting Pep2-8 hydrogels are biocompatible, 3-dimensional polymers with tunable properties. Small hydrogel therapies offer advantages over currently marketed biologics. They can reduce the yearly cost of LDL-c treatment with biologics to a price that can be realistically prescribed to millions of patients with high cholesterol. The hydrogels are composed of a polymeric network and are mechanically stronger and less soluble than serums or dissolved solutions.

Unlike orally administered drugs, which require additional tuning and processing to enable the drug material to pass through biological barriers in the mouth, the hydrogels are delivered subcutaneously, much closer to the target site, improving pharmacokinetics. No additional processing is generally needed to allow drug material to pass through barriers. Hydrogels are, therefore, much easier to design; but administration as not as user-friendly. Drug decomposition is also a factor during development. Large and small molecule drugs that enter the body as pills or injected serums are often quickly degraded by proteases and enzymes. Degradation impacts the intended biological function of drug molecules, decreasing efficacy. The hydrogels are composed of a polymeric network and are mechanically stronger and less soluble than serums or dissolved solutions. When the hydrogel is injected as an implant, the crosslinks between the polymers break down slowly, releasing the drug material steadily over time. This is beneficial from both a dosing perspective (less spikes and rapid declines in performance) and from a pharmacodynamic perspective (biochemical dynamics are more consistent).

The hydrogels are easy and inexpensive to formulate, water-based, completely biocompatible, and have tunable properties. The disclosed hydrogel peptides are initially a non-viscous liquid, enabling them to be homogenously diluted to 2% weight (or any concentration) in sucrose and brought to a neutral pH. When hydrogelation is induced, the peptide becomes viscous and gel-like and exhibits shear-thinning. This enables to peptide to be easily loaded into and injected from the dosing syringe as non-viscous liquid. Once in the body, it regains its viscous properties and is slowly released as a Pep2-8 drug molecule.

Accordingly, the disclosed therapy can be delivered as a hydrogel, which offers several benefits over small-molecule oral delivery, including controlled release, tunable delivery properties and lack of need for oral availability. Additionally, the disclosed method combines the affordability of statins with the decreased rate of adverse effects associated with PCKS9 drugs. PCKS9 inhibition more directly targets the CVD pathology pathway.

In accordance with the disclosure, Pep2-8 is formulated as a hydrogel by attaching its 13 amino-acid sequence (TVFTSWEEYLDWV) to a multidomain peptide of mainly ABA block structure (ESLSLSLSLSLSLEG) to generate the full sequence (ESLSLSLSLSLSLEGTVFTSWEEYLDWV). The polymer was synthesized, recovered by continuous flow dialysis (to increase dialysis yield) and sterile filtered into an instrument that lyophilizes the sample. During hydrogel formulation, the polymer was dissolved into sucrose and the pH was brought to 7.0 with the addition of NaOH. The multidomain of the peptide was then neutralized by adding positive ions (CaCl2). Neutralizing the multidomain enables the polymer to aggregate into nanofibers, ultimately forming a hydrogel.

The addition of the multidomain peptide allows hydrogel formation. The B domain contains the charged residue Lysine (+) which maintains electrostatic repulsion at a neutral pH, preventing self-assembly. When the lysines are neutralized, in the presence of negatively charged ions, the peptides self-assemble into nanofibers and, ultimately, into a hydrogel. Serine in the A domain is associated with higher mechanical strength and the ability to exhibit shear-thinning with mechanical recovery when force is removed.

Non limiting exemplary embodiments of the disclosure are as follows:

Paragraph A: A self-assembled, multidomain, peptide-based hydrogel capable of inhibiting serine protease for reduction of cholesterol levels, comprising a first domain, a second domain, third domain, and a fourth domain wherein: the first domain is (X)n where X is a negatively or positively charged amino acid, and the magnitude of n is less than or equal to 4, wherein the first domain is positioned at both the N-terminal and the C-terminal of the second domain; the second domain is (YZ)n′ where Y is a hydrophilic amino acid and Z is a hydrophobic amino acid or where Y is a hydrophobic amino acid and Z is a hydrophilic amino acid and n′ is 2 to 7; the third domain is a spacer; and the fourth domain is a bioactive peptide sequence.

Paragraph B: The composition of Paragraph A wherein X is selected from the group consisting of glutamic acid, aspartic acid, arginine, histidine, and lysine.

Paragraph C: The composition of Paragraph A wherein the second domain hydrophobic amino acid is selected from the group consisting of alanine, valine, leucine, glycine, isoleucine, tryptophan, tyrosine, phenylalanine, proline, methionine, and cysteine; and the second domain hydrophilic amino acid is selected from the group consisting of serine, tyrosine, threonine, asparagine, and glutamine.

Paragraph D: The composition of Paragraph A wherein Y is serine and Z is leucine and n′ is 6.

Paragraph E: The composition of Paragraph A wherein the spacer is selected from the group consisting of aminohexanoic acid, polyethylene glycol, and 5 or fewer glycine residues.

Paragraph F: The composition of Paragraph A wherein the bioactive peptide sequence is a combination of amino acids that inhibits serine protease for the reduction of cholesterol levels.

Paragraph G: The composition of Paragraph A further comprising a buffer wherein the buffer comprises negatively-charged ions when X is a positively-charged amino acid and comprises positively-charged ions when X is a negatively-charged amino acid, and wherein the peptide is at final concentration from about 0.10 mg/mL to about 100 mg/mL.

Paragraph H: The composition of Paragraph F wherein the final concentration of the peptide is greater than 0.10 mg/mL and less than or equal to 100 mg/mL wherein the peptide has an initial storage modulus at 1% strain, wherein the initial storage modulus is greater than 90% recoverable within about 5 minutes following exposure to shearing at 100% strain for one minute.

Paragraph I: The self-assembled multidomain peptide-based hydrogel of claim 1, wherein the first, second, and third domain comprises (ESLSLSLSLSLSLEG), wherein E is Glutamic Acid, S is Serine, L is Leucine, and G is Glycine and wherein the fourth domain comprises (TVFTSWEEYLDWV), wherein T is Threonine, V is Valine, F is Phenylalanine, S is Serine, W is Tryptophan, E is Glutamic Acid, Y is Tyrosine, L in Leucine, and D is Aspartic Acid.

Paragraph J: The composition of Paragraph H wherein the peptide is in solution at a concentration from about 0.10 mg/mL to about 100 mg/mL, wherein the solution comprises sucrose, and wherein the composition further comprises a buffer having positively-charged ions, wherein the ratio of the buffer to the solution is 1:40.

Paragraph K: A method comprising: administering a composition as provided in Paragraphs F, G, H and I to a target location of a subject and allowing the composition to form a hydrogel scaffold at the target location following administration.

Paragraph L: The method of Paragraph K where the step of administering the composition is performed by injection.

Paragraph M: The method of Paragraph K wherein the final concentration of the peptide in the composition is from about greater than 0.10 mg/mL to about 100 mg/mL.

Paragraph N: The method of Paragraph K wherein the final concentration of the peptide in the composition is 20 mg/mL.

Paragraph O: The method of Paragraph K wherein the composition is capable of inhibiting serine protease for reduction of cholesterol levels.

Paragraph P: The method of Paragraph N wherein the serine protease comprises proprotein convertase subtilisin/kexin type 9.

Paragraph Q: The method of Paragraph K wherein the composition is a pharmaceutically effective amount of the peptide-based hydrogel.

Paragraph R: The method of Paragraph K wherein the patient is suffering from high-cholesterol or from symptoms attributed to cardiovascular disease.

Paragraph S: The method of Paragraph K wherein the composition is administered in addition to other small or large molecule therapies for the reduction of high-cholesterol or other symptoms attributed to cardiovascular disease.

Paragraph T: A method of inhibiting serine protease for reducing cholesterol levels, comprising: administering through injection or placement of a pharmaceutically effective amount of a multidomain peptide-based hydrogel comprising the amino acid sequence (ESLSLSLSLSLSLEGTVFTSWEEYLDWV), wherein T is Threonine, V is Valine, F is Phenylalanine, S is Serine, W is Tryptophan, E is Glutamic Acid, Y is Tyrosine, L is Leucine, D is Aspartic acid, and G is Glycine.

Paragraph U: The self-assembled multidomain peptide-based hydrogel of Paragraph A, wherein the serine protease comprises proprotein convertase subtilisin/kexin type 9.

Paragraph V: A self-assembled multidomain peptide-based hydrogel capable of inhibiting serine protease comprising the amino acid sequence (ESLSLSLSLSLSLEGTVFTSWEEYLDWV), wherein T is Threonine, V is Valine, F is Phenylalanine, S is Serine, W is Tryptophan, E is Glutamic Acid, Y is Tyrosine, L in Leucine, D is Aspartic Acid, and G is Glycine.

The above objects, advantages and non-limiting exemplary embodiments of the disclosure are met by the presently disclosed multidomain peptide-based hydrogels. In addition, the above and yet other objects and advantages of the present invention will become apparent from the hereinafter-set forth Brief Description of the Drawings, Detailed Description of the Invention, and claims appended herewith. These features and other features are described and shown in the following drawings and detailed description.

### BRIEF DESCRIPTION OF THE DRAWINGS

So that those having ordinary skill in the art will have a better understanding of how to make and use the disclosed composition and methods, reference is made to the accompanying figures wherein:

FIG. 1 is a prior art pictorial illustration of PCKS9 binding to a LDL-c receptor and how less LDL receptors remain since the receptor and bond LDL-c are destroyed; and

FIG. 2 is a pictorial illustration of subcutaneous injection of the Pep2-8 hydrogel to a target organ site for inhibition of proprotein convertase subtilisin/kexin type 9 for lowering cholesterol levels.

### DETAILED DESCRIPTION

Low-density lipoproteins (LDL) make up the majority of the cholesterol found in the body. As previously discussed, LDL receptors mediate endocytosis of LDL, diminishing the level of LDL in the blood plasma to homeostasis. After internalization, the ligand dissociates and the receptor folds back and recycles onto the cell surface, making itself available to bind to more LDL molecules. This activity is in part modulated by proprotein convertase subtilisin/kexin type 9 (PCSK9), an inhibitory enzyme that binds with the EGF-A domain of the LDL receptor and prevents the conformational change of the receptor-ligand complex. This causes the natural intracellular degradation of the receptor, preventing recycling onto the cell surface. As a result, the cholesterol level increases in the blood stream causing hypercholesterolemia.

Several peptides are known to mimic the EFG-A domain of the LDL receptor and bind with PCSK9, inhibiting its action and allowing the receptor to perform its normal function. However, the delivery of a small peptide is difficult without a proper vehicle, since small peptides are rapidly cleared in the body. This disclosure presents an injectable, peptide-based hydrogel delivery system that incorporates a peptide inhibitor of PCSK9. The self-assembling peptide system is thixotropic and forms a hydrogel with high epitope presentation of the PCSK9 inhibitor. The result is a hydrogel that has better targetability and persistence and can activate more receptors. The disclosure contemplates a library of clinically relevant LDL lowering peptide hydrogel drugs that can be tailored for (i) decreased dosing, (ii) increased compliance, (iii) decreased immunogenicity compared to the standard-of-care monoclonal antibody Evolucimab® that would ensure long term utility of the therapeutic, and (iv) lower cost compared to the standard-of-care.

The hydrogel is a polymer composed of the repeating multidomain peptide (MDP) sequence (ESLSLSLSLSLSLEGTVFTSWEEYLDWV). Specifically, Pep2-8 is formulated as a hydrogel by attaching its 13 amino-acid sequence (TVFTSWEEYLDWV) to a self-assembling peptide of mainly an ABA block structure (ESLSLSLSLSLSLEG) to generate the full sequence (ESLSLSLSLSLSLEGTVFTSWEEYLDWV). In the 13 amino-acid sequence: T is Threonine, V is Valine, F is Phenylalanine, S is Serine, W is Tryptophan, E is Glutamic Acid, Y is Tyrosine, L in Leucine, and D is Aspartic Acid.

ABA block refers to a block copolymer structure of a polypeptide. B represents the core structure and A represents the tail ends. The As and Bs represent polypeptide/amino acid structures. In the disclosure, the ABA structure is mainly the noted ABA structure (ESLSLSLSLSLSLEG) where the amino acids are: E is Glutamic Acid, S is Serine, L is Leucine, and G is Glycine. The A Block in the above disclosure is E or a Glutamic Acid Block. The core structure B Block is SLSLSLSLSLSL or a Serine-Leucine block. The G at the end is Glycine and used as a glycine spacer between block sequences that can be modified to incorporate other functional moieties to promote peptide solubility and activity.

The first portion of the sequence (ESLSLSLSLSLSLEG) is responsible for hydrogelation and contains two domains: the termini (charged amino acids on both ends of the sequence) and the midblock (alternating hydrophobic and hydrophilic residues). When the polymer is dissolved in sucrose at neutral pH, the charged amino acids in the termini promote solubility and generate electrostatic, repulsive forces between monomers, preventing self-assembly.

The addition of the self-assembling peptide allows hydrogel formation. When the polymer is neutralized in the presence of positively charged ions, the peptides self-assemble into nanofibers and, ultimately, into the hydrogel. Serine in the A domain is associated with higher mechanical strength and the ability to exhibit shear-thinning with mechanical recovery when force is removed.

The addition of positive calcium ions neutralizes the charged amino acids and forms cross-links at termini locations. Cross-links between monomers create a self-assembling polymer chain that forms a nanofiber. Each nanofiber consists of a bilayer, peptide ‘sandwich’ in which the hydrophobic portions of the midblock face inward, to minimize water contact, the hydrophilic portions of the midblock face outward and the termini cross-link with adjacent peptides in the chain. Hydrogen bonds also form between nanofibers, further stabilizing the hydrogel. The second portion of the MDP sequence (TVFTSWEEYLDWV) is responsible for drug activity and contains one domain: the signaling domain (in this case, the molecule Pep2-8).

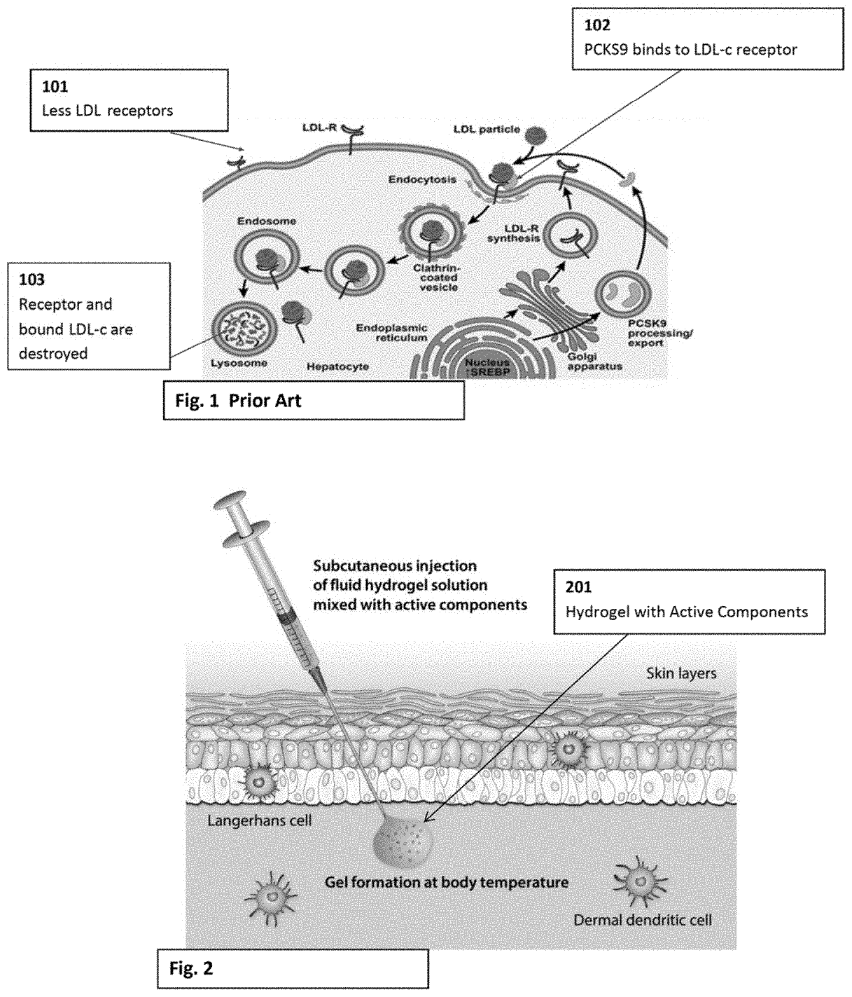

Adverting to the Figures, FIG. 1 is from Lambert et al, Journal of Lipid Resources, vol. 53: 2515-2524 (2012), and illustrates the problem caused by PCSK9 binding to LDL-c receptors. As shown in FIG. 1, block 101 refers to how there are less LDL-r or LDL receptors on the surface. This figure shows the explanation of how as in block 102 PCKS9 binds to a LDL-c receptor and in block 103 both the receptor and the bound LDL-c are later destroyed resulting in less LDL receptors.

FIG. 2 illustrates one embodiment of administration of the present hydrogel with active components that prevents the cycle shown in FIG. 1. As shown in block 201 the hydrogel with the active components is administered subcutaneously. A gel formation at body temperature is formed below skin layers. The hydrogel is designed to carry the active components that disrupt the cycle shown in FIG. 1.

### EXAMPLE 1.

To facilitate a better understanding of the present invention, the following example of specific instances is given. In no way should the following example be read to limit or define the entire scope of the invention. The following materials and methods were employed for the Example below.

The polymer synthesis took place in a small-scale laboratory. The full sequence peptide was synthesized, recovered by continuous flow dialysis (to increase dialysis yield), and sterile filtered into an instrument that lyophilizes the sample. After lyophilization, the peptide was dissolved into sucrose at 2% wt. (4 mg for every 200 μL sucrose; yields a 6.18 mM solution) and the pH was brought to 7.0 with the addition of 0.5% NaOH at 1.0 μL increments.

During dialysis, synthesized protein was placed in a selectively permeable membrane that draws out contaminants, leaving the purified protein behind. After dialysis, the protein was placed in a lyophilizer to dry. The mass of the dried product was the final yield, normally 50-100 mg. The protein yield was increased by several mg by constructing a continuous flow dialysis setup that maintained a larger concentration gradient, drawing out more contaminants and enabling more protein to be retrieved.

The multidomain of the peptide was then neutralized by adding positive calcium ions from aqueous calcium chloride (0.62M CaCl2; 5 μL CaCl2 per 200 μL sucrose). The ratio of calcium chloride to sucrose was determined by conducting a molar charge balance between calcium (Ca2+) and the charge of polymer (−5). Neutralizing the multidomain enabled the polymer to aggregate into nanofibers, ultimately forming a hydrogel. The neutralizing volume of calcium chloride was significantly smaller than the bulk volume of sucrose and polymer (1:40) to limit the volume of extraneous water and chloride in the hydrogel solution.

The polymer was kept sterile at all stages to prevent bacteria, fungus and other contaminants from entering the sample by monitoring polymer sterility with fibroblasts. If fibroblasts exposed to the hydrogel polymer died or showed bacterial/fungal growth, the sample had been compromised. In an initial fibroblast test with the hydrogel, the cells showed bacterial growth under the microscope. To prevent bacteria from entering the sample (as well as other contaminants), the polymer product was sterile filtered in the lyophilizer, as well as all solvents that were exposed to the polymer during hydrogel formulation. The polymer and hydrogel were handled and prepared in a biohood. In addition, the hydrogel was UV sterilized overnight before in vivo or in vitro tests. After incorporating these steps, the fibroblasts did not show any sign of bacterial growth or contaminants, suggesting hydrogel was ready for experimental testing.

During formulation, the Pep2-8 hydrogel formed a gel initially and would liquify with application of shear force (as expected) but would not return to a gel when the force was removed. Shear-thinning with recovery is an important characteristic of a hydrogel. This was achieved by developing a mathematical model for the charge balance of the polymer in sucrose for determining that the addition of NaOH during the pH step allowing nanofibers formation.

Shear thinning with recovery enables the hydrogel to be easily loaded into and injected subcutaneously from a dosing syringe as non-viscous liquid. Once in the body, it regains its viscous properties and slowly dissolves into the blood stream. The hydrogel multidomain is, therefore, the delivery vehicle for the active drug ingredient, Pep2-8. All preparation and loading steps were carried out in a biosafety cabinet to maintain sterility of the hydrogel. The gel was, additionally, exposed to UV light overnight before loading into the dosing syringe as an extra precaution.

The multidomain peptides (MDP) synthesized pursuant to this disclosure are short amino acids sequences with repeating hydrophobic and hydrophilic motifs that can be triggered to self-assemble in aqueous solution to form β-sheets and long-range nanofibers. The MDP sequence contains three previously described domains: the termini (charged amino acid residues), the midblock (alternating hydrophobic and hydrophilic residues) and the signaling domain. These residues can be switched with similar residues. Self-assembly is mediated by bonds that break and reassemble quickly: hydrogen bonding, Van der Waal's interactions, and ionic interactions. This affords thixotropic rheological properties—rapid shear thinning and shear recovery. Therefore, these hydrogels can be easily syringe aspirated, injected, and re-assemble in situ to provide a prolonged, sustained response which has been evaluated for drug delivery and angiogenesis. At the ultrastructural level, MDP self-assembles into large-scale extracellular matrix (ECM) mimetic nanofibers 2 nm thick, 6 nm wide, and nm to μm long. Injectable ECM mimetic scaffolds may rapidly infiltrate with cells that loaded drug can phenotypically modulate. Building upon in vivo drug release, the signaling domain (Pep2-8) was engineered into the peptide sequence to allow for the competitive binding of PCSK9, preventing PCSK9 from binding to LDL receptors and allowing them to recycle back to the surface of the cells to bind more LDL.

The protocol and Pep2-8 hydrogels were verified by several analytical methods. Rheology, differential scanning calorimetry and mass spectroscopy on the hydrogel were conducted. These methods confirmed the hydrogels have the required composition, shear thinning and recovery characteristics.

### Experimental Section

It is contemplated that the effects of the disclosed PCSK9 regulating, multidomain, peptide-based hydrogel can be assessed with an in-vivo mouse model and in-vitro fibroblast and hepatocyte model. In all conditions, the mice or cells will be exposed to the Pep2-8 hydrogel. The hydrogel will be formulated by synthesizing full sequence polymer, recovering protein with dialysis, lyophilizing protein to remove all water, dissolving protein in sucrose at 2% weight, bringing solution to neutral pH with addition of 0.5% NaOH, and neutralizing charge of sequence, to induce hydrogel formation, with addition of 0.62M CaCl2. Fibroblast cells will be exposed to the Pep2-8 hydrogel in culture media to test for hydrogel toxicity. Hepatocytes can be monitored to determine if the hydrogel increases LDL receptor expression on the cell surface and decreases LDL-c in media. Mice can be monitored to determine if the hydrogel has any cholesterol lowering effects.

### Contemplated In Vivo and In Vitro Dosage and Cholesterol Effects-In-Vivo

It is contemplated that mice will be maintained on a western diet to mimic the diet typically consumed in Europe and the United States. Mice is put on the western diet 4 weeks prior to testing.
1. Normal laboratory mice eat, on average, slightly less than 4 g/day (average 3.5 g with some studies showing males at 3.75 g; this number is not for lactating, weaning or rapid growth mice)
2. Provide enough food (in grams) of the test western diet for mice to eat for a week ad libitum
a. (6 mice*4 g/day*7 days)=168 g
b. Food and bedding will preferably be changed once per week, if weight gain is unstable it may be helpful to change food twice per week. Researchers have reported this can help stabilize fluctuating data on high-fat diets.
3. Maintain for 8 week duration of study

The multidomain peptide based hydrogel will be administered to mice (n=6) under four different dosage conditions and their blood cholesterol concentration will be monitored according to the chart below. Cholesterol concentrations will be determined using a commercially available cholesterol assay kit.

It is contemplated that the mice fed a western diet will have higher cholesterol levels than control mice fed with normal chow. The western diet mice will be injected with Pep2-8 hydrogel. The Pep2-8 hydrogel is expected to bind to circulating PCSK9, ultimately decreasing blood cholesterol. The experiments as shown in Table 1 are intended to indicate (1) decreased blood cholesterol with Pep2-8 hydrogel compared to controls and (2) dosage information for sustained/significant cholesterol reduction.

TABLE 1
Experimental Design

| Week | 1 | 2 | 3 | 4 | 5 | 6 | 7 | 8 |
|---|---|---|---|---|---|---|---|---|
| Group 1 | 50 μL inj. + | 50 μL inj. + | 50 μL inj. + | 50 μL inj. + | Blood Draw | Blood Draw | Blood Draw | Blood Draw |
| | Blood Draw | Blood Draw | Blood Draw | Blood Draw | | | | |
| Group 2 | 200 μL inj. + | 200 μL inj. + | 200 μL inj. + | 200 μL inj. + | Blood Draw | Blood Draw | Blood Draw | Blood Draw |
| | Blood Draw | Blood Draw | Blood Draw | Blood Draw | | | | |
| Group 3 | 50 μL inj. | Blood Draw | Blood Draw | Blood Draw | Blood Draw | Blood Draw | Blood Draw | Blood Draw |
| | Blood Draw | | | | | | | |
| Group 4 | 200 μL inj. | Blood Draw | Blood Draw | Blood Draw | Blood Draw | Blood Draw | Blood Draw | Blood Draw |
| | Blood Draw | | | | | | | |

### In Vitro

### Hydrogel Toxicity

The Pep2-8 hydrogel will be tested for toxicity by exposing various concentrations of the 2% weight hydrogel (0.618 mM; 0.0618 mM; 0.00618 mM) to fibroblast cells. The concentrations listed are the molar concentrations of the sequenced peptide at 2% weight in sucrose across three sequential 10× dilutions. The Pep2-8 hydrogel is introduced on day 1, after cells are passaged and confluent. The cells will be maintained for 7 days and toxicity is assessed by optical microscopy (check bacteria, fungus, etc.) and by fluorescent live/dead staining.

### Hepatocyte LDL Receptor Expression and LDL Uptake

The Pep2-8 hydrogel will be tested for efficacy by exposing various concentrations of the 2% weight hydrogel (6.18 mM; 0.618 mM; 0.0618 mM; 0.00618 mM) to hepG2 cells, pre-incubated with PCSK9. After 4-hours of hydrogel exposure, the cells will be tested for LDL receptor expression. Additionally, a separate group of hepatocytes will be exposed to hydrogel and PCSK9 for 1.5 hours. A fluorescence assay will be run on LDL cholesterol to determine the concentration of cholesterol post hydrogel exposure. It is contemplated that the Pep2-8 hydrogel will increase LDL receptor expression on hepatocytes and decrease cholesterol concentration in media compared to controls.

It is contemplated that the Pep2-8 hydrogel, when exposed to with hepatocytes cultured with PCSK9, will bind to and inhibit the convertase. PCSK9 inhibition should increase the recycling rate of LDL receptors to the surface of the cells, thereby increasing cholesterol digestion and decreasing the level of cholesterol contained in the media. These experiments should indicate (1) greater LDL receptor expression and (2) lower cholesterol in media with Pep2-8 hydrogel exposure compared to controls.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A self-assembled, multidomain, peptide-based hydrogel capable of inhibiting serine protease for reduction of cholesterol levels, comprising a first domain, a second domain, third domain, and a fourth domain wherein:
the first domain is (X)ₙ where X is a negatively or positively charged amino acid, and the magnitude of n is less than or equal to 4, wherein the first domain is positioned at both the N-terminal and the C-terminal of the second domain;
the second domain is (YZ)_{n′} where Y is a hydrophilic amino acid and Z is a hydrophobic amino acid or where Y is a hydrophobic amino acid and Z is a hydrophilic amino acid and n′ is 2 to 7;
the third domain is a spacer; and
the fourth domain is a bioactive peptide sequence.

2. The composition of claim 1 wherein X is selected from the group consisting of glutamic acid, aspartic acid, arginine, histidine, and lysine.

3. The composition of claim 1 wherein the second domain hydrophobic amino acid is selected from the group consisting of alanine, valine, leucine, glycine, isoleucine, tryptophan, tyrosine, phenylalanine, proline, methionine, and cysteine; and the second domain hydrophilic amino acid is selected from the group consisting of serine, tyrosine, threonine, asparagine, and glutamine.

4. The composition of claim 1 wherein Y is serine and Z is leucine and n′ is 6.

5. The composition of claim 1 wherein the spacer is selected from the group consisting of aminohexanoic acid, polyethylene glycol, and 5 or fewer glycine residues.

6. The composition of claim 1 wherein the bioactive peptide sequence is a combination of amino acids that inhibits serine protease for the reduction of cholesterol levels.

7. The composition of claim 1 further comprising a buffer wherein the buffer comprises negatively-charged ions when X is a positively-charged amino acid and comprises positively-charged ions when X is a negatively-charged amino acid, and wherein the peptide is at final concentration from about 0.10 mg/mL to about 100 mg/mL.

8. The composition of claim 6 wherein the final concentration of the peptide is greater than 0.10 mg/mL and less than or equal to 100 mg/mL wherein the peptide has an initial storage modulus at 1% strain, wherein the initial storage modulus is greater than 90% recoverable within about 5 minutes following exposure to shearing at 100% strain for one minute.

9. The self-assembled multidomain peptide-based hydrogel of claim 1, wherein the first, second, and third domain comprises (ESLSLSLSLSLSLEG), wherein E is Glutamic Acid, S is Serine, L is Leucine, and G is Glycine and wherein the fourth domain comprises (TVFTSWEEYLDWV), wherein T is Threonine, V is Valine, F is Phenylalanine, S is Serine, W is Tryptophan, E is Glutamic Acid, Y is Tyrosine, L in Leucine, and D is Aspartic Acid.

10. The composition of claim 8 wherein the peptide is in solution at a concentration from about 0.10 mg/mL to about 100 mg/mL, wherein the solution comprises sucrose, and wherein the composition further comprises a buffer having positively-charged ions, wherein the ratio of the buffer to the solution is 1:40.

11. A method comprising: administering a composition as provided in claim 6 to a target location of a subject and allowing the composition to form a hydrogel scaffold at the target location following administration.

12. The method of claim 11 where the step of administering the composition is performed by injection.

13. The method of claim 11 wherein the final concentration of the peptide in the composition is from about greater than 0.10 mg/mL to about 100 mg/mL.

14. The method of claim 11 wherein the final concentration of the peptide in the composition is 20 mg/mL.

15. The method of claim 11 wherein the composition is capable of inhibiting serine protease for reduction of cholesterol levels.

16. The method of claim 14 wherein the serine protease comprises proprotein convertase subtilisin/kexin type 9.

17. The method of claim 11 wherein the composition is a pharmaceutically effective amount of the peptide-based hydrogel.

18. The method of claim 11 wherein the patient is suffering from high-cholesterol or from symptoms attributed to cardiovascular disease.

19. The method of claim 11 wherein the composition is administered in addition to other small or large molecule therapies for the reduction of high-cholesterol or other symptoms attributed to cardiovascular disease.

20. A method of inhibiting serine protease for reducing cholesterol levels, comprising: administering through injection or placement of a pharmaceutically effective amount of a multidomain peptide-based hydrogel comprising the amino acid sequence (ESLSLSLSLSLSLEGTVFTSWEEYLDWV), wherein T is Threonine, V is Valine, F is Phenylalanine, S is Serine, W is Tryptophan, E is Glutamic Acid, Y is Tyrosine, L is Leucine, D is Aspartic acid, and G is Glycine.

21. The self-assembled multidomain peptide-based hydrogel of claim 1, wherein the serine protease comprises proprotein convertase subtilisin/kexin type 9.

22. A self-assembled multidomain peptide-based hydrogel capable of inhibiting serine protease comprising the amino acid sequence (ESLSLSLSLSLSLEGTVFTSWEEYLDWV), wherein T is Threonine, V is Valine, F is Phenylalanine, S is Serine, W is Tryptophan, E is Glutamic Acid, Y is Tyrosine, L in Leucine, D is Aspartic Acid, and G is Glycine.

23. The method of claim 11 comprising: administering a composition as provided in claim 7 to a target location of a subject and allowing the composition to form a hydrogel scaffold at the target location following administration.

24. The method of claim 11 comprising: administering a composition as provided in claim 8 to a target location of a subject and allowing the composition to form a hydrogel scaffold at the target location following administration.

25. The method of claim 11 comprising: administering a composition as provided in claim 9 to a target location of a subject and allowing the composition to form a hydrogel scaffold at the target location following administration.


SEQUENCE LISTING (1 sequence)

SEQ ID NO: 1 - protein, 28 aa, Artificial Sequence
  Pep2-8
ESLSLSLSLSLSLEGTVFTSWEEYLDWV